(19) United States
(12) Patent Application Publication
(10) Pub. No.: US 2009/0137808 A1
(43) Pub. Date: May 28, 2009

(54) PROCESS FOR MANUFACTURING BISPHOSPHONIC ACIDS

(75) Inventors: Edward G. Samsel, Baton Rouge, LA (US); Tse-Chong Wu, Baton Rouge, LA (US)

Correspondence Address: ALBEMARLE CORPORATION;PATENT DEPARTMENT, 451 FLORIDA STREET, BATON ROUGE, LA, 70801, US

(73) Assignee: ALBEMARLE CORPORATION, Baton Rouge, LA (US)

(21) Appl. No.: 12/282,725

(22) PCT Filed: Mar. 16, 2007
(86) PCT No.: PCT/US07/64176

§ 371 Date: Sep. 12, 2008

Related U.S. Application Data
(60) Provisional application No. 60/784,752, filed on Mar. 21, 2006.

(51) Int. Cl. C07D 221/02; C07D 233/54; C07D 213/02; C07F 9/38

(52) U.S. Cl. 546/23; 548/112; 546/22; 562/13

(57) ABSTRACT

A manufacturing process for the preparation of bisphosphonic acids and in particular zoledronic acid is provided wherein diglyme, monoglyme, or a mixture thereof, is utilized to produce a homogenous, water soluble, solid reaction mass that upon cooling, dissolving in water and stripping results in a high purity product and comparatively good yield. wherein Ri is selected from the group consisting of

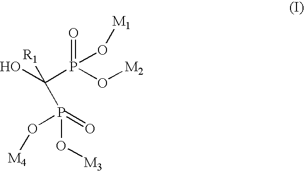

### BACKGROUND OF THE INVENTION

The present invention relates to an improved industrial process for the preparation of bisphosphonic acids and their pharmacologically active salts, and in particular, 1-hydroxy-2-(imidazol-1-yl)ethylidene-1,1-bisphosphonic acid, commonly referred to as zoledronic acid. The bisphosphonic acids described herein are suitable for the treatment of diseases of the skeletal system and in cases when bone formation and/or calcium metabolism have been disturbed, such as in the therapy of bone metastases.

The bisphosphonic acids described herein have the following structure:

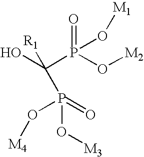

wherein M1, M2, M3 and M4 are selected from hydrogen and a monovalent cation and R1 can be one of the following:

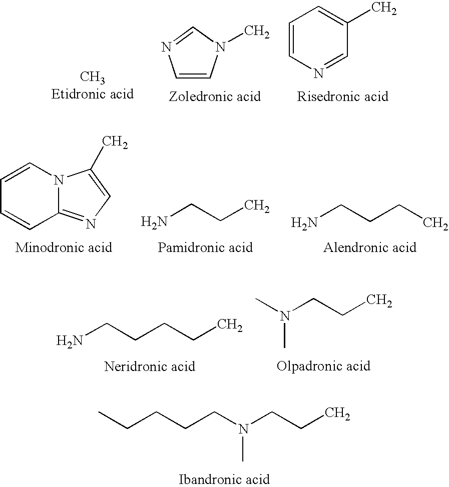

U.S. Pat. Nos. 4,939,130 and 4,777,163 disclose a process for making bisphosphonic acids based upon a known method published by Kabachnick et al. [Izv. Akad. Nauk. USSR, Ser. Khim., 2, 433-437, (1987)]. The synthesis basically consists of reacting the appropriate w-amino acid with a mixture of phosphorous acid and one of the three phosphorus chlorides, phosphorus trichloride, phosphorus oxychloride, or phosphorus pentachloride, then quenching the reaction mixture with water or a non-oxidizing aqueous acid followed by heating to hydrolyse the phosphorous intermediate to the final product.

One problem associated with this process involves the solidification of the reaction melt. The reaction starts as a two-phase system that gradually thickens into a non-stirrable mass. This problem was acknowledged in CA Patent No. 2,018,477 and 2,044,923 wherein the inventors utilized methanesulfonic acid to solubilize the reaction components and keep it fluid up to completion of the reaction. Unfortunately, methanesulfonic acid reacts with phosphorus trichloride and under adiabatic conditions the reaction becomes self-heating at 85° C. and an uncontrolled exotherm occurs at >140° C.

Others have tried to address the solidification problem utilizing various solvent systems. For example, U.S. Pat. No. 6,201,148 utilizes N-protected derivatives and phosphoric acid as a solvent; U.S. Pat. No. 6,573,401 describes the use of methanesulfonic anhydride; and published US Patent Application No. 2005288509 describes the use of ionic solvents comprising ammonium, sulphonium or phosphonium salts. However, these systems present various drawbacks including safety concerns, high costs, product contamination, and/or additional processing steps.

Another example of a prior art solvent system that addresses the solidification problem during the preparation of alendronic acid is U.S. Pat. No. 5,908,959 which describes a process utilizing a high molecular weight polyalkylene glycol, or its derivatives. However, when employed with the process of the present invention, the yield and purity were unsatisfactorily low.

### SUMMARY OF THE INVENTION

Accordingly, the present invention provides a manufacturing process for the preparation of bisphosphonic acids and in particular zoledronic acid. While the description that follows relates specifically to the manufacture of zoledronic acid, the process may be easily adapted to manufacture other bisphosphonic acids by selecting the appropriate starting materials.

### DETAILED DESCRIPTION OF THE INVENTION

The first step in the manufacturing process is the preparation of t-butyl imidazoleacetate from imidazole and t-butyl chloroacetate, which is described in U.S. Pat. No. 4,584,008 and is incorporated herein by reference in its entirety to the extent allowed by applicable law.

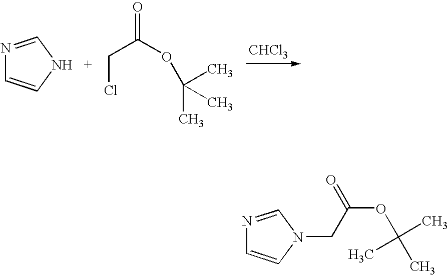

Reaction temperature may range from about 0° C. to about 100° C., or from about 50° C. to about 70° C. The reaction mass may be stirred and/or refluxed from about 1 to about 24 hours. Generally, from about 0.5 to 5 moles, or from about 2 to 3 moles, of the imidazole is used per mole of t-butyl chloroacetate. The reaction takes place in a suitable inert inorganic solvent, for example, chloroform. Other suitable inert organic solvents that can be used for this step include, for example, methylene chloride, carbon tetrachloride, benzene, toluene, and the like and compatible mixtures thereof.

Upon completion, the reaction mass is cooled to about ambient temperature and the organic phase is extracted, washed, and stripped under reduced pressure to yield t-butyl imidazole-1 acetate.

The second step in the manufacturing process is the hydrolysis of t-butyl imidazole-1 acetate to imidazole-1 acetic acid.

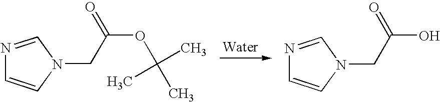

The t-butyl imidazole-1 acetate is hydrolyzed by dissolving in about 20 to about 40, or in about 30 to about 35, molar equivalents of water and heating to about 100° C. The byproduct, t-butanol, is driven off and upon cooling the reaction mixture to about ambient temperature and stripping of the reaction mixture under vacuum, imidazole-1 acetic acid remains as a solid product.

The phosphonation of imidazole-1 acetic acid is the final step of the process and the step in which the above-described solidification problem occurs. Rather than addressing the solidification problem by attempting to maintain the continuous solubility of the reaction mass, either monoglyme (1,2-dimethoxy ethane), diglyme (bis(2-methoxyehthyl)ether), or a mixture thereof, is utilized to create a homogeneous solid that can be easily penetrated with water.

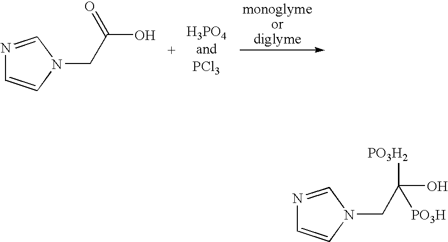

The imidazole-1 acetic acid is combined with between about 1 and about 5, or between about 2 and about 4, molar equivalents of phosphorus trichloride and between about 1 to about 2 molar equivalents of phosphoric acid. A stoichiometric amount of phosphoric acid can be used. The reactants are combined in a sufficient volume of monoglyme or digylme to ensure the imidazole-1 acetic acid is substantially dissolved, for example about 1 to about 5 molar equivalents, or about 2 and about 4 molar equivalents. The reaction mass is stirred at a controlled temperature of between about 40° C. and about 80° C. until the evolution of hydrogen chloride ceases, after which the reaction mass is stirred at a higher temperature, for example between about 60° C. and about 90° C. At the higher temperature, a solid homogeneous mass forms that can no longer be stirred, but is heated further, for example, for about 1 to about 10 hours, to maximize yield.

The homogenous mass is allowed to cool, e.g., to about ambient temperature or below. Water is then slowly added to dissolve the homogenous mass after which the solution is refluxed, cooled, stripped and re-dissolved in water until all solids are dissolved. Zoledronic acid may then be collected from the resulting solution by conventional means, i.e. seeded crystallization.

### EXAMPLE 1

### Preparation of Imidazoleacetic Acid

A 50 L reactor was charged with chloroform (54 kg), imidazole (6.13 kg, 90.04 mol) and t-butyl chloroacetate (5.48 kg, 36.4 mol). The temperature was increased to 60° C. over a 2 hour period and maintained at 60° C. for an additional 24 hours. The reaction mass was cooled to room temperature. The chloroform phase was washed successively with four portions of water (7.2 kg each) to remove imidazolium salts and excess imidazole.

Water (15.1 kg) was added and chloroform was removed by distillation with a jacket temperature of 60-65° C. (53° C. is the boiling point of the azeotrope) using a Dean-Stark trap to return the water phase. After chloroform was removed, the reactor jacket temperature was slowly raised to 115° C. during which time t-butanol and water co-distilled (azeotrope boiling point is 80° C.). After the alcohol was removed, the aqueous solution was cooled and drained from the reactor, giving 17.54 kg of solution containing imidazoleacetic acid (3.77 kg, 29.9 mol, 82.2% yield as assayed by NMR).

### EXAMPLE 2

### Isolation of Imidazoleacetic Acid

A portion of the solution from Example 1 (1.13 kg) was rotary evaporated to give a slurry of solids (0.38 kg) to which was added acetone (234 g) to complete crystallization. The solid was filtered, washed with acetone and dried with a stream of nitrogen. The evaporator condensate was re-evaporated, washed and dried to give a second crop of crystals; this was combined with the first, to give imidazoleacetic acid (219 g, 91% recovery, 98.9 wt % pure by NMR assay). ¹H NMR (D20): 8.68 (s, 1H); 7.42 (s, 2H); 4.83 (s, 2H); 4.79 (br s, 1H).

### EXAMPLE 3

### Preparation of Zoledronic Acid

A 1.5 liter kettle reactor, fitted with a heating mantle, mechanical stirrer, dropping funnel, thermocouple and condenser with nitrogen inlet adapter, was charged with imidazoleacetic acid (100 g, 0.793 mol), diglyme (400 ml), and 85% phosphoric acid (55 ml). Phosphorus trichloride (330 g, 2.41 mol) was slowly added to the reaction mass resulting in an exotherm and the evolution of hydrogen chloride. The temperature was allowed to rise to 70° C. and the solution was stirred until the evolution of HCl subsided. The temperature of the reaction mass was increased to 85° C. and a white solid began to form, float and adhere to the stirrer shaft. After about 1 hour, stirring became impossible and the stirring motor was stopped. The reaction mass was heated for 5 more hours at 85° C. and then cooled to ambient temperature, producing a solid homogeneous white mass.

Water was slowly added to the white mass (320 ml) that resulted in an exotherm and HCl evolution. The water slowly dissolved the mass in a gradual and uniform fashion, eventually liberating the stirrer. After the mass substantially dissolved, the solution was refluxed for 5 hours, then cooled and stripped to a gum with a rotary evaporator, collecting 420 g of water (pH 0.65). More water (250 ml) was added and stripped, collecting 166 g of water (pH 1.87). Water (250 ml) was again added and stripped, collecting 316 g (pH 2.14). The flask was removed from the rotary evaporator, water (150 ml) was added and the mixture was heated to 90-95° C. during which time all solids dissolved. The solution was seeded with zoledronic acid monohydrate crystals and slowly cooled to room temperature then chilled to 3° C. with an ice bath. The resulting crystalline solid was filtered, rinsed with acetone (200+100 ml) and dried under a nitrogen stream giving a crop of 52.4 g. Acetone was also added to the filtrate (200 ml) and the solution was left in a freezer overnight giving a second crop of crystals (12.0 g) which, after washing with acetone and drying, was combined with the first crop for a total yield of 64.4 g (28%). The NMR indicated the presence of traces of diglyme, acetone and H₃PO₃ impurities.

### EXAMPLE 4

### Preparation of Zoledronic Acid

A 5 liter cylindrical jacketed reactor was fitted with a mechanical stirrer, thermocouple, nitrogen inlet adapter and a condenser with a caustic scrubber. This was charged with imidazoleacetic acid (0.333 kg, 2.64 mol) and diglyme (1.00 l). The slurry was heated to 50° C. while stirring (100 rpm) under a slow nitrogen purge (1 l/min). Additional diglyme (0.26 l) and 85% phosphoric acid (0.304 kg) were added to the reaction mass. Using a Masterflex pump and Teflon tubing, phosphorus trichloride (1.04 kg total, 7.57 mol) was pumped into the reaction mass, slowly (2 ml/min) at first and then at an increased rate (40 ml/min), after the water in the phosphoric acid had been depleted. During addition of the PCl₃, the temperature was raised to about 65° C. and a white mass gradually formed, causing the stirrer to bind. The jacket temperature was increased to 85° C. causing PCl₃ to reflux. The refluxing slowed and then stopped as the white mass expanded. The reactor was allowed to stand at about 80° C. for four hours, after which the jacket temperature was set at 15° C. overnight.

The reactor jacket temperature was increased to 50° C. and water (0.95 kg total) was slowly (2-5 ml/min) added with a Masterflex pump. The water dissolved the white mass on contact, liberating HCl in an exothermic reaction. After about 250 g of water was added to the reaction mass, the stirrer became unbound and stirring was resumed (100 rpm). The water addition rate was slowly increased to 40 ml/min. The reaction mass was then heated at about 100° C. for 4 hours and then cooled to room temperature.

The reaction mass was drained and rotary evaporated to yield a gum. Water was added to the gum and stripped several times until the distillates pH rose above 1. The resulting aqueous solution (1.2 kg, 1.6 l) was stirred in a beaker and acetone (1.5 l) was slowly added. The mixture was allowed to stand 16 hours to complete crystallization. The solid was filtered, thoroughly washed with acetone and dried in a nitrogen stream to give crude Zoledronic acid (0.202 g, 0.74 mol. 28% yield).

### EXAMPLE 5

### Recrystallization of Zoledronic Acid Monohydrate

A jacketed 3 liter flask, fitted with a stirrer, thermocouple and nitrogen adapter was charged with water (1.5 l) and 64.4 g of crude zoledronic acid monohydrate. The aqueous mixture was heated to 85° C. and all solids dissolved giving a pH of 1.7. Absolute ethanol (500 ml) and zoledronic acid monohydrate seeds were added to the aqueous mixture creating a slurry, which was slowly cooled with stirring. At 38° C., the pH was adjusted from 3.7 to 1.7 with hydrochloric acid. At 18° C., the aqueous mixture was adjusted to pH greater than 2. The slurry was stirred at 0° C. for about 4 hours then the solid was filtered, washed with ethanol (2×200 ml) and dried with nitrogen yielding 58.64 g of zoledronic acid monohydrate. The product was dried further in a vacuum drying oven at 50° C., 1-2 in. nitrogen, giving a loss of 0.28 wt %. An NMR assay indicated a product purity of 92.2 wt % (on an anhydrous basis). Karl-Fischer titration indicated 6.46% water corresponding to 98.7% zoledronic acid hydrate with the water to a zoledronic mole ratio of 1.06:1. ¹H NMR (D2O/NaOD): 7.75 (s, 1H); 7.23 (s, 1H); 6.90 (s, 1H); 4.82 (O—H, 7.35H); 4.46 (m, 2H); ³¹P (H coupled, D2O/NaOD): 16.83 (m).

### EXAMPLE 6

### (Comparative) Preparation of Zoledronic Acid in PEG-400

Example 3 was repeated substituting PEG-400 (400 ml) for diglyme. After the addition of phosphorus trichloride and increased temperature of the reaction mass, a solid formed that eventually returned to solution upon further heating. The yield of zoledronic acid was 7% (isolated yield). ¹HNMR (D₂O/NaOD): 7.72 (s, 1H); 7.22 (s, 1H); 6.87 (s, 1H); 4.82 (O—H, 7.02H); 4.45 (m, 2H). ³¹P NMR (D₂O/NaOD): 17.0 (m). Not only was there a substantial decrease in yield, but the product purity deteriorated as well.

While the compositions and methods of this invention have been described in terms of preferred embodiments, it will be apparent to those of skill in the art that variations may be applied to the compositions, methods and/or processes and in the steps or in the sequence of steps of the methods described herein without departing from the concept and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the scope and concept of the invention.

What is claimed is:

1. A process for preparing bisphosphonic acids and their salts having the structure 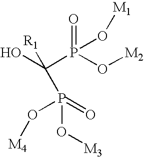
wherein R₁ is selected from the group consisting of 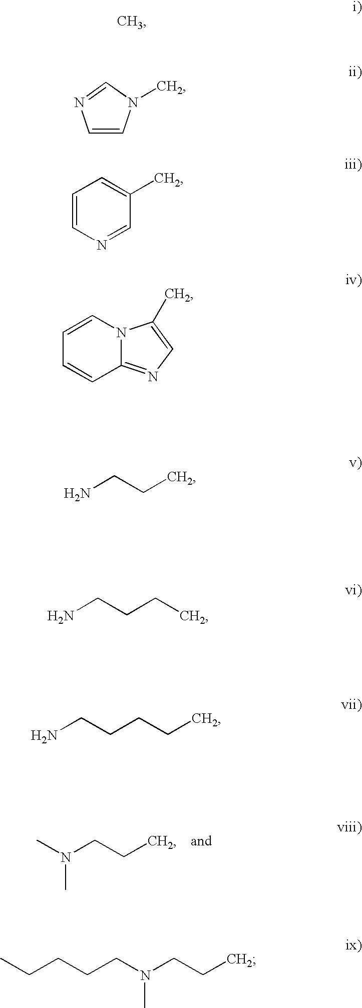
and M₁, M₂, M₃, and M₄ are selected from the group consisting of hydrogen and a monovalent cation, the process comprising the reaction of acids R₁—CO₂H, wherein R₁ is as previously described, with an acid selected from phosphoric acid, phosphorus acid, or a mixture of phosphoric and phosphorus acids, and phosphorus trichloride, characterized in that the reaction is conducted in diglyme, monoglyme, or a combination of diglyme and monoglyme.

2. A process for preparing zoledronic acid and its salts comprising the reaction of imidazoleacetic acid with an acid selected from phosphoric acid, phosphorus acid, or a mixture of phosphoric and phosphorus acids, and phosphorus trichloride, characterized in that the reaction is conducted in diglyme, monoglyme, or a combination of diglyme and monoglyme.

3. The process of claim 2, wherein the reaction is carried out at a temperature of from about 40° C. to about 90° C. and is characterized by the formation of a water soluble solid.

4. The process of claim 2, wherein the reaction is carried out at an initial reaction temperature of from about 40° C. to about 80° C. and is increased to a subsequent reaction temperature of from about 60° C. to about 90° C., and is characterized by the formation of a water soluble solid.

5. The process of claim 4, wherein the subsequent reaction temperature is maintained for about 1 to about 10 hours.

6. The process of claim 5, wherein the amount of phosphorus trichloride ranges between about 1 and about 5 molar equivalents.

7. The process of claim 6, wherein the amount of phosphorus trichloride ranges between about 2 and about 4 molar equivalents.

8. The process of claim 5, wherein a stoichiometric amount of acid is utilized.

9. The process of claim 5 further comprising cooling the water soluble solid to or below ambient temperature, adding water to dissolve the water soluble solid, and collecting the zoledronic acid product.
